(19) United States
(12) Patent Application Publication
(10) Pub. No.: US 2023/0110193 A1
(43) Pub. Date: Apr. 13, 2023

(54) METHODS FOR THE TREATMENT OF HEADACHE DISORDERS

(71) Applicant: Boehringer Ingelheim International GmbH, Ingelheim am Rhein (DE)

(72) Inventors: Michael Paul PIEPER, Biberach an der Riss (DE); Stefan KASPERS, Mainz (DE)

(21) Appl. No.: 17/913,271

(22) PCT Filed: Apr. 6, 2021
(86) PCT No.: PCT/EP2021/058857

§ 371 Date: Sep. 21, 2022

(30) Foreign Application Priority Data
Apr. 7, 2020 (EP) 20168355.4

(51) Int. Cl. A61K 31/7048; A61K 45/06; A61P 25/06

(52) U.S. Cl. CPC A61K 31/7048; A61K 45/06; A61P 25/06

(57) ABSTRACT

SGLT2 inhibitors, in particular empagliflozin, can be used in methods for the treatment of headache disorders.

### FIELD OF THE INVENTION

This invention relates to SGLT2 inhibitors, in particular empagliflozin, for use in methods for the treatment of headache disorders, in particular primary headache disorders like migraine, tension-type headache and trigeminal autonomic cephalalgias, optionally in combination with other pharmaceutically active substances. In addition, the invention relates to pharmaceutical compositions comprising said inhibitors and optionally other pharmaceutically active substances and to methods for the treatment of headache disorders with said inhibitors or compositions, optionally in combination with other pharmaceutically active substances.

### BACKGROUND OF THE INVENTION

Headache disorders characterized by recurrent headache are among the most common disorders of the nervous system, with a high worldwide prevalence in particular among adults. Headache itself is a painful and disabling feature of a number of primary headache disorders, namely migraine, tension-type headache, and trigeminal autonomic cephalalgias.

Not only is headache painful, but it is also disabling; headache disorders are one of the major causes worldwide of years lost due to disability. In particular, migraine and tension-type headache are of public health importance since they are responsible for high population levels of disability and ill-health and thus result in a high socioeconomic burden. Headache disorders impose a recognizable burden on sufferers including sometimes substantial personal suffering, impaired quality of life and financial cost. Repeated headache attacks, and often the constant fear of the next one, damage family life, social life and employment. The long-term effort of coping with a chronic headache disorder may also predispose the individual to other illnesses.

The pathophysiology of migraine is not yet fully understood. Genetic predisposition and environmental triggers appear important for the pathogenesis of migraine. Migraine is often described as a neurovascular disorder. It involves neuronal as well as vascular mechanisms which are relevant and probably interrelated. Also, metabolic mechanisms in migraine pathophysiology have been postulated.

Migraine most often begins in puberty and mostly affects those aged between 35 and 45 years. It is more common in women, usually by a factor of about 2:1, presumably because of hormonal influences. Migraine is recurrent, often life-long, and characterized by periodic attacks. Attacks typically include one-sided headache of moderate to severe intensity which is pulsating in quality and aggravated by routine physical activity. Visual disturbance, nausea, vomiting and sensitivity to light, sound or smell may be associated features. Typically, untreated migraine attacks have a duration of 4-72 hours and their frequency is anywhere between once a year and once a week.

Appropriate treatment of headache disorders requires training of health professionals, accurate diagnosis and recognition of the conditions, appropriate treatment with cost-effective medications, simple lifestyle modifications, and patient education. Although some migraine patients can be cured with life style modification (trigger elimination) and can be treated with over-the-counter medications or by acupuncture, hypnosis or the like, the majority of patients are in the need of prescription drugs for relief from the migraine and prevention of further attacks. The symptoms most in need of treatment are the head pain and gastrointestinal symptoms. Photophobia and the aura may also have to be treated.

Pharmacological headache disorder treatment, especially migraine treatment, often includes both preventive therapy, aiming at reducing attack frequency and severity, and acute therapy, for aborting attacks. The main classes of drugs to treat headache disorders include non-specific drugs (like analgesics and non-steroidal anti-inflammatory drugs) and specific drugs (like specific anti-migraine medications such as ergot derivatives and triptans); antiemetic or prokinetic drugs may be co-administered to facilitate absorption of the primary drug and especially for headache attacks accompanied by nausea and vomiting; it is also important to avoid chronification of the headache and the development of medication-overuse headache.

Still, there is a high unmet medical need to explore potential novel treatment options for patients with headache disorders syndrome.

SGLT2 inhibitors are compounds inhibiting sodium/glucose cotransporter 2 (SGLT2). They are used in particular in the treatment of diabetes mellitus. Examples for marketed SGLT2 inhibitors are empagliflozin, canagliflozin, dapagliflozin, ertugliflozin, ipragliflozin, luseogliflozin, remogliflozin etabonate, sergliflozin etabonate, sotagliflozin and tofogliflozin.

Empagliflozin is the compound 1-chloro-4-(β-D-glucopyranos-1-yl)-2-[4-((S)-tetrahydrofuran-3-yloxy)-benzyl]-benzene of the formula

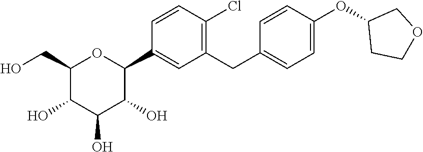 [SMILES: OC[C@H]1O[C@@H](c2ccc(Cl)c(Cc3ccc(O[C@H]4CCOC4)cc3)c2)[C@H](O)[C@@H](O)[C@@H]1O]

as described for example in WO 2005/092877. Methods for its synthesis are known to the one skilled in the art and are also described in the literature, for example in WO 2006/120208, WO 2007/031548 and WO 2011/039108. An advantageous crystalline form of empagliflozin is described in WO 2006/117359 and WO 2011/039107 which hereby are incorporated herein in their entirety. This crystalline form possesses good solubility properties which enables a good bioavailability of the SGLT2 inhibitor. Furthermore, the crystalline form is physico-chemically stable and thus provides a good shelf-life stability of the pharmaceutical composition. Preferred pharmaceutical compositions, such as solid formulations for oral administration, for example tablets, are described in WO 2010/092126, which hereby is incorporated herein in its entirety.

Empagliflozin is a potent and selective inhibitor of SGLT2, i.e. it reduces the reabsorption of glucose and sodium in the kidneys, thus lowering blood sugar and making it useful in the treatment of e.g. type 2 diabetes mellitus. In addition, empagliflozin has been shown in clinical studies as well as on a preclinical level to provide cardiovascular benefits, to modulate blood pressure, to increase free fatty acids and to moderately increase blood ketone body levels.

### SUMMARY OF THE INVENTION

In a first aspect, the present invention relates to SGLT2 inhibitors for use in a method for the treatment of headache disorders.

In a second aspect, the present invention relates to a pharmaceutical composition comprising one or more of said inhibitors and one or more pharmaceutically acceptable excipients for use in a method for the treatment of headache disorders.

In a third aspect, the present invention relates to a method for the treatment of headache disorders in a patient in need thereof, the method being characterized in that one or more of said inhibitors is administered to the patient.

In a fourth aspect, the present invention relates to the use of one or more of said inhibitors in the manufacture of a medicament for the treatment of headache disorders in a patient in need thereof.

Further aspects of the present invention will become apparent to the person skilled in the art directly from the foregoing and following description and the examples.

### General Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

Within this invention, the term “empagliflozin” also comprises its hydrates, solvates and polymorphic forms thereof, as well as prodrugs thereof, e.g. esters or carbonate esters, in particular etabonates, which are hydrolyzed in vivo to the pharmacologically active compound. The same applies to the term “SGLT2 inhibitors” in general as well as to the INN names of the other specific SGLT2 inhibitors mentioned within this invention.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making organic or inorganic acid or base salts thereof. Examples of pharmaceutically acceptable salts include, but are not limited to, mineral or organic acid salts of basic residues such as amines; alkali or organic salts of acidic residues such as carboxylic acids; and the like.

Salts of other acids than those mentioned above which for example are useful for purifying or isolating the compounds of the present invention (e.g. trifluoro acetate salts) also comprise a part of the invention.

The term “tablet” comprises tablets without a coating and tablets with one or more coatings. Furthermore, it comprises tablets having one, two, three or even more layers and press-coated tablets, wherein each of the beforementioned types of tablets may be without or with one or more coatings. The term “tablet” also comprises mini, melt, chewable, effervescent and orally disintegrating tablets.

All the doses or dosage units of a physiologically acceptable salt of one of the above-mentioned active compounds should be understood as being doses or dosages of the active compound itself.

The terms “combination” or “combined” within the meaning of this invention may include, without being limited, fixed and non-fixed (e.g. free) forms (including kits) and uses, such as e.g. the simultaneous, sequential or separate use of the components or ingredients.

The terms “treatment” and “treating” as used herein embrace both therapeutic, i.e. curative and/or palliative, especially abortive and/or acute, treatment and preventive, i.e. prophylactic, treatment.

Therapeutic treatment (“therapy”) refers to the treatment of patients having already developed one or more of said conditions in manifest, acute or chronic form. Therapeutic treatment may be symptomatic treatment in order to relieve the symptoms of the specific indication or causal treatment in order to reverse or partially reverse the conditions of the indication or to stop or slow down progression of the disease.

Preventive treatment (“prevention”, “prophylaxis”) refers to the treatment of patients at risk of developing one or more of said conditions, prior to the clinical onset of the disease in order to reduce said risk.

The terms “treatment” and “treating” include the administration of one or more active compounds, in particular therapeutically effective amounts thereof, in order to prevent or delay the onset of the symptoms or complications and to prevent or delay the development of the disease, condition or disorder and/or in order to eliminate or control the disease, condition or disorder as well as to alleviate the symptoms or complications associated with the disease, condition or disorder.

The term “therapeutically effective amount” means an amount of a compound of the present invention that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

When this invention refers to patients in need of treatment, it relates primarily to treatment in mammals, in particular humans. Human patients may be adults, adolescents or children. Adult patients are of the age of 18 years or older. Adolescents are of age 10 to 17 years, preferably of age 13 to 17 years. Children are of age 2 to 10 years, preferably of age 6 to 10 years.

Within this invention, the definitions and classification of headache disorders are followed as given by the Headache Classification Committee of the International Headache Society (IHS) in The International Classification of Headache Disorders, 3rd edition (ICHD-3) (Cephalalgia 2018, 38(1), 1-211), which is hereby incorporated in its entirety. In particular, the terms defining medical conditions like “headache disorders”, “primary headache disorders”, “migraine”, “tension-type headache (TTH)”, “trigeminal autonomic cephalalgias (TACs)”, “other primary headache disorders” as well as any subclasses thereof are used and are to be interpreted in line with ICHD-3.

### DETAILED DESCRIPTION OF THE INVENTION

The present invention allows for an efficient treatment of headache disorders with manageable side effects in patients by administration of SGLT2 inhibitors.

In a first aspect of the present invention, it is found that SGLT2 inhibitors may be beneficial for the treatment of headache disorders. This may be rationalized by the fact that, for instance, the SGLT2 inhibitor empagliflozin is able to address a number of pathophysiological principles e.g. underlying migraine.

The pathophysiology of migraine has been described for a long time to have a vascular component.

Empagliflozin has vascular effects as is evident e.g. from its effect on the body's salt and water metabolism, from its blood pressure modulating properties, and from its proven cardiovascular benefit shown in clinical trials (Zinman et al., N Engl J Med (2015); 373(22):2117-28) as well as on a preclinical level (Park et al., Cardiovasc Diabetol (2020); 19(1):19). These features may thus also contribute to beneficial effects of empagliflozin in the treatment of patients with migraine.

Further, migraine is known to be associated with insulin resistance and metabolic syndrome, which may be interpreted such that these disorders at least partially share a common etiology, be it related to inflammation or to defects in glucose metabolism. Further, oxidative stress is discussed to be causatively related to migraine.

Empagliflozin has been described to have positive effects on glucose metabolism (Ferrannini et al., Diabetes Obes Metab (2013), 15(8):721-8; Zinman et al., N Engl J Med (2015), 373(22):2117-28) and also anti-inflammatory effects (Benetti et al., J Pharmacol Exp Ther (2016), 359(1):45-53; Iannantuoni et al., J Clin Med (2019), 8(11); Amin et al., Fundam Clin Pharmacol (2020), 34(5):548-558). Further, beneficial effects reducing reactive oxygen species have been reported (Uthman et al., Cell Physiol Biochem (2019), 53(5):865-886; Das et al., Cell Signal (2020), 68:109506; Amin et al., Fundam Clin Pharmacol (2020), 34(5):548-558) as well as on mitochondrial function (Mizuno et al., Physiol Rep (2018), 6(12):e13741; Shao et al., Cardiovasc Diabetol (2019), 18(1):165). Further, beneficial effects on intracellular sodium (Na+) levels are described for empagliflozin (Baartscheer et al., Diabetologia (2017), 60(3):568-573; Bertero et al., Cardiovasc Res (2018), 114(1):12-18). Accordingly, it also has a positive impact on insulin resistance and metabolic syndrome (Kern et al., Metabolism (2016), 65(2):114-23; Xu et al., BMJ Open Diabetes Res Care (2019), 7(1):e000783). Thus, similarly, benefits may be obtained in the treatment of migraine with empagliflozin. Moderately elevated levels of ketone bodies in the blood have been reported to have a variety of potentially beneficial effects in migraine pathophysiology and can protect against migraine, e.g. as observable in patients on a ketogenic diet.

This positive effect may also be achieved by empagliflozin leading to increased levels of ketone bodies. A moderate increase of ketone bodies following the intake of empagliflozin has been shown in several clinical trials and in preclinical studies (Ferrannini et al., Diabetes Care (2016); 39(7):1108-14; Kim et al., Diabetes Obes Metab (2019); 21(4):801-811).

Positive effects of empagliflozin administration relevant for the treatment of migraine may also be corroborated by preclinical studies which are used to investigate aspects of anti-migraine activity.

Although it remains to be elucidated what the exact underlying mechanisms responsible for the empagliflozin activity are, these findings are expected to translate into clinically beneficial effects in the treatment of migraine patients.

This view finds support in observations from clinical trials which indicate that empagliflozin may in fact decrease the number of migraine attacks, as well as in case reports on reduced migraine frequency in patients on treatment with other SGLT2 inhibitors.

Therefore, the administration of SGLT2 inhibitors may be clinically beneficial for the treatment of migraine and other headache disorders.

The benefits of the treatment with SGLT2 inhibitors may come into effect for different types of headache disorders, especially for primary headache disorders like migraine, tension-type headache (TTH), trigeminal autonomic cephalalgias (TACs) and other primary headache disorders, including all subtypes and subclasses of conditions comprised by these terms according to ICHD-3, in particular for migraine, migraine without aura, migraine with aura, chronic migraine, complications of migraine (e.g. status migrainosus), probable migraine, and episodic syndromes that may be associated with migraine.

The treatment of headache disorders with SGLT2 inhibitors may be preventive and/or therapeutic, e.g. acute; in particular for migraine, also abortive treatment is desirable.

Preferred SGLT2 inhibitors are those that are already marketed in commercial drug products for other indications as they are expected to meet the safety and tolerability requirements also for the headache disorder treatment, in particular empagliflozin, canagliflozin, dapagliflozin, ertugliflozin, ipragliflozin, luseogliflozin, remogliflozin, sergliflozin, sotagliflozin and tofogliflozin, most preferably empagliflozin. Also, prodrugs of said inhibitors may be used, in particular the carbonate esters thereof, more specifically the etabonates thereof, e.g. remogliflozin etabonate or sergliflozin etabonate.

Thus, according to one embodiment of the first aspect of the present invention, an SGLT2 inhibitor, in particular selected from the group consisting of empagliflozin, canagliflozin, dapagliflozin, ertugliflozin, ipragliflozin, luseogliflozin, remogliflozin, sergliflozin, sotagliflozin and tofogliflozin and the carbonate ester prodrugs thereof, in particular empagliflozin, is provided for use in a method for the treatment of headache disorders.

According to another embodiment, said inhibitor is preferably provided for use in a method for the treatment of primary headache disorders, selected from migraine, tension-type headache (TTH), trigeminal autonomic cephalalgias (TACs) and other primary headache disorders, more preferably for migraine.

According to another embodiment, said inhibitor is more preferably provided for use in a method for the treatment of migraine, selected from migraine without aura, migraine with aura, chronic migraine, complications of migraine (e.g. status migrainosus), probable migraine, and episodic syndromes that may be associated with migraine.

According to another embodiment, said inhibitor is provided for use in a method for the treatment of tension-type headache (TTH), selected from infrequent and frequent episodic TTH, chronic TTH and probable TTH.

According to another embodiment, said inhibitor is provided for use in a method for the treatment of trigeminal autonomic cephalalgias (TACs), selected from cluster headache, paroxysmal hemicrania, short-lasting unilateral neuralgiform headache attacks, hemicrania continua and probable TAC.

According to another embodiment, said inhibitor is provided for use in a method for the treatment of other primary headaches, selected from primary cough headache, primary exercise headache, primary headache associated with sexual activity, primary thunderclap headache, cold-stimulus headache, external-pressure headache, primary stabbing headache, nummular headache, hypnic headache and new daily persistent headache.

According to another embodiment, said inhibitor is provided for use in a method for the preventive treatment of the above-mentioned headache disorders.

According to another embodiment, said inhibitor is provided for use in a method for the therapeutic, e.g. the acute, treatment of the above-mentioned headache disorders, in particular for the abortive treatment of migraine.

The above-mentioned SGLT2 inhibitors for use in the treatment of headache disorders are administered in therapeutically effective amounts. Typically these are achieved by total daily doses ranging from 1 mg to 300 mg, in particular from 1 mg to 25 mg per day, e.g. 1 mg, 2.5 mg, 5 mg, 10 mg, 12.5 mg, 15 mg, 25 mg, 100 mg and 300 mg, preferably 2.5 mg, 5 mg, 10 mg and 25 mg.

The actual therapeutically effective amount or therapeutic dosage will of course depend on factors known by those skilled in the art such as age and weight of the patient, route of administration and severity of disease. In any case, the active compound will be administered at dosages and in a manner which allows a therapeutically effective amount to be delivered based upon a patient's unique condition. Likewise, the determination of the necessity of dose adjustments, e.g. due to adverse reactions to the active pharmaceutical ingredient, and their putting into practice will be known to the one skilled in the art.

The administration may be once, twice or thrice daily, preferably once daily, in particular in the case of preventive treatment.

The route of administration is oral, buccal, sublingual, nasal, parenteral, inhalative, transdermal, subcutaneous, intravenous, intramuscular, rectal, topical, intraocular, intravitreal or intraperitoneal, preferably oral.

Suitable doses, administration schemes and formulations of the exemplary SGLT2 inhibitors mentioned above are known to the one skilled in the art.

Thus, according to one embodiment of the first aspect of the present invention, the SGLT2 inhibitor for use as defined hereinbefore or hereinafter is provided for once-daily oral administration in a dose ranging from 1 mg to 25 mg.

According to a preferred embodiment, empagliflozin is administered orally, e.g. in the form of a tablet, in a total daily dose ranging from 1 mg to 25 mg, preferably once daily.

For the treatment of the above-mentioned headache disorders, the SGLT2 inhibitors may optionally be used in combination with one or more other pharmaceutically active substances suitable for the preventive and/or therapeutic, e.g. acute, treatment of headache disorders, either simultaneously or sequentially. The dose strengths, administration schemes and formulations of these other substances, in particular of those specifically mentioned hereinafter, are known to the one skilled in the art. The combination with more than one other active medicaments is useful, for example, when an SGLT2 inhibitor combined with another suitable pharmaceutically active substance has a synergistic effect against pain, but at the same time an antiemetic activity is also desired.

The combined administration may be together or separate, simultaneous or sequential, joint or time-shifted, e.g. in one single or in more than one separate formulations or dosage forms, wherein the administration of one element of the combination may be prior to, concurrent to, or subsequent to the administration of the other element of the combination. The combination treatment may be a first line, second line or third line therapy, or initial or add-on combination therapy or replacement therapy. Advantageously, the number and/or dosage, i.e. dose strength and/or frequency, of other pharmaceutically active substance of the combination are reduced while the administration of empagliflozin is initiated and/or continued.

The SGLT2 inhibitors may also be used in combination with nutritional supplements, e.g. coenzyme Q10, magnesium oxide, and riboflavin.

Also, the SGLT2 inhibitors may optionally be used in combination with non-medication based treatment approaches like lifestyle changes, muscle relaxation techniques, acupuncture, intra-oral appliances, surgery, neuromodulation or neurostimulation, e.g. sphenopalatine ganglion stimulation, single-pulse transcranial magnetic stimulation, noninvasive vagus nerve stimulation, transcutaneous supraorbital nerve stimulation.

Pharmaceutically active substances suitable for the treatment of headache disorders are, for example, beta adrenergic blockers (e.g. acebutolol, atenolol, betaxolol, bisoprolol, metoprolol, nadolol, nebivolol, propanolol, timolol),

antiepileptics or anticonvulsants (e.g. sodium valproate, valproic acid, divalproex sodium, topiramate, carbamazepine, pregabalin, gabapentin, phenytoin, vigabatrin, levetiracetam),
antidepressants, especially tricyclic antidepressants or selective serotonin reuptake inhibitors, (e.g. amitriptyline, bupropion, venlafaxine, imipramine, fluoxetine, duloxetine, citalopram, escitalopram, fluvoxamine, paroxetine, sertraline, trazodone),
Ca channel antagonists (e.g. amlodipine, felodipine, isradipine, lacidipine, lercanidipine, manidipine, nicardipine, nifedipine, nilvadipine, nimodipine, nisoldipine, nitrendipine, flunarizine, diltiazem, gallopamil, verapamil),
calcitonine gene-related peptide (CGRP) antagonists (e.g. ubrogepant, rimegepant, atogepant),
monoclonal antibodies against CGRP and its receptor (e.g. eptinezumab, galcanezumab, fremanezumab, erenumab) monoclonal antibodies against pituitary adenylate cyclase-activating polypeptide (PACAP 38) and its receptor PAC1 (e.g. AMG-301, ALD1910), and
angiotensin-II antagonists (e.g. azilsartan, candesartan, eprosartan, irbesartan, losartan, olmesartan, tasosartan,
telmisartan, valsartan),
histamine-H1-receptor antagonists (e.g. cyproheptadine, diphenhydramine, promethazine),
alpha adrenergic agonists (e.g. clonidine, guanfacine, tizanidine),
alpha adrenergic antagonists (e.g. indoramine), NO-synthase inhibitors, neuroleptics, and further pharmaceutically active substances (e.g. botulinum toxin, methysergide, methylergometrine, memantine), analgesics and non-steroidal anti-inflammatory drugs (NSAIDs) (e.g. acemetacin, acetylsalicylic acid, azathioprine, ibuprofen, fenoprofen, flurbiprofen, ketoprofen, dexketoprofen, fenbufen, naproxen, naproxen sodium, lornoxicam, meloxicam, piroxicam, tenoxicam, acetaminophen, caffeine, acclofenac, diclofenac, diclofenac potassium, diclofenac epolamine, zomepirac, indomethacin, ketorolac, etodolac, tolfenamic acid, metamizole (dipyrone), phenazone, pethidine, dextropropoxyphen, diflunisal, leflunomide, mefenamic acid, phenylbutazone, sulphasalazine, celecoxib, etoricoxib, parecoxib, rofecoxib, valdecoxib and, as well as combinations thereof, especially the combination of acetylsalicylic acid and/or acetaminophen with caffeine, as well as substances which inhibit the earlier or later stages of prostaglandin synthesis, or prostaglandin receptor antagonists, such as for example EP2-receptor antagonists and IP-receptor antagonists)
ergot derivatives (e.g. ergotamine, ergotamine tartrate, optionally in combination with caffeine, dihydroergotamine), serotonin 5-HT_{1B/1D} agonists, in particular triptans, (e.g. sumatriptan, zolmitriptan, naratriptan, rizatriptan, eletriptan, frovatriptan, almotriptan),
serotonin 5-HT_{1F} agonists (e.g. lasmiditan),
glutamate receptor antagonists (e.g. tezampanel),
CGRP antagonists (e.g. ubrogepant, rimegepant, atogepant),
antiemetic or prokinetic drugs (e.g. domperidone, metoclopramide, prochlorperazine, chlorpromazine, dimenhydrinate, cyclizine, droperidol, haloperidol),
corticosteroids (e.g. dexamethasone),
antimuscarinics, AMPA antagonists, neurokinin antagonists,
and further pharmaceutically active substances (e.g. lidocaine (lignocaine), pizotifen, lisinopril, isometheptene)
or the physiologically acceptable salts thereof as well as the combinations thereof.

Such other pharmaceutically active substances suitable in particular for preventive treatment may be, for example, beta adrenergic blockers (e.g. acebutolol, atenolol, betaxolol, bisoprolol, metoprolol, nadolol, nebivolol, propanolol, timolol),

antiepileptics or anticonvulsants (e.g. sodium valproate, valproic acid, divalproex sodium, topiramate, carbamazepine, pregabalin, gabapentin, phenytoin, vigabatrin, levetiracetam),
antidepressants, especially tricyclic antidepressants or selective serotonin reuptake inhibitors, (e.g. amitriptyline, bupropion, venlafaxine, imipramine, fluoxetine, duloxetine, citalopram, escitalopram, fluvoxamine, paroxetine, sertraline, trazodone),
Ca channel antagonists (e.g. amlodipine, felodipine, isradipine, lacidipine, lercanidipine, manidipine, nicardipine, nifedipine, nilvadipine, nimodipine, nisoldipine, nitrendipine, flunarizine, diltiazem, gallopamil, verapamil), calcitonine gene-related peptide (CGRP) antagonists (e.g. ubrogepant, rimegepant, atogepant),
monoclonal antibodies against CGRP and its receptor (e.g. eptinezumab, galcanezumab, fremanezumab, erenumab) and further pharmaceutically active substances (e.g. botulinum toxin, methysergide, methylergometrine, memantine, acetylsalicylic acid),
monoclonal antibodies against pituitary adenylate cyclase-activating polypeptide (PACAP 38) and its receptor PAC1 (e.g. AMG-301, ALD1910), and
angiotensin-II antagonists (e.g. azilsartan, candesartan, eprosartan, irbesartan, losartan, olmesartan, tasosartan, telmisartan, valsartan),
histamine-H1-receptor antagonists (e.g. cyproheptadine),
alpha adrenergic agonists (e.g. clonidine, guanfacine, tizanidine),
alpha adrenergic antagonists (e.g. indoramine), NO-synthase inhibitors, neuroleptics,
and further pharmaceutically active substances (e.g. pizotifen, lisinopril)
or the physiologically acceptable salts thereof as well as the combinations thereof.

Preferably such other substances for preventive treatment are propanolol, timolol, metoprolol, atenolol, nadolol, divalproex sodium, topiramate, gabapentin, amitriptylin, bupropion, fluoxetin, nimodipine, flunarizine, rimegepant, atogepant, eptinezumab, galcanezumab, fremanezumab, erenumab, botulinum toxin and verapamil,

most preferably propanolol, timolol, metoprolol, divalproex sodium, topiramate, gabapentin, amitriptyline, flunarizine, galcanezumab, fremanezumab and erenumab.

Such other pharmaceutically active substances suitable in particular for therapeutic, e.g. acute, treatment may be, for example,

analgesics and non-steroidal anti-inflammatory drugs (NSAIDs) (e.g. acemetacin, acetylsalicylic acid, azathioprine, ibuprofen, fenoprofen, flurbiprofen, ketoprofen, dexketoprofen, fenbufen, naproxen, naproxen sodium, lornoxicam, meloxicam, piroxicam, tenoxicam, acetaminophen, caffeine, acclofenac, diclofenac, diclofenac potassium, diclofenac epolamine, zomepirac, indomethacin, ketorolac, etodolac, tolfenamic acid, metamizole (dipyrone), phenazone, pethidine, dextropropoxyphen, diflunisal, leflunomide, mefenamic acid, phenylbutazone, sulphasalazine, celecoxib, etoricoxib, parecoxib, rofecoxib, valdecoxib and, as well as combinations thereof, especially the combination of acetylsalicylic acid and/or acetaminophen with caffeine, as well as substances which inhibit the earlier or later stages of prostaglandin synthesis, or prostaglandin receptor antagonists, such as for example EP2-receptor antagonists and IP-receptor antagonists)
ergot derivatives (e.g. ergotamine, ergotamine tartrate, optionally in combination with caffeine, dihydroergotamine), serotonin 5-HT_{1B/1D} agonists, in particular triptanes, (e.g. sumatriptan, zolmitriptan, naratriptan, rizatriptan, eletriptan, frovatriptan, almotriptan, avitriptan),
serotonin 5-HT_{1F} agonists (e.g. lasmiditan),
glutamate receptor antagonists (e.g. tezampanel),
CGRP antagonists (e.g. ubrogepant, rimegepant, atogepant),
antiemetic or prokinetic drugs (e.g. domperidone, metoclopramide, prochlorperazine, chlorpromazine, dimenhydrinate, cyclizine, droperidol, haloperidol),
histamine-H1-receptor antagonists (e.g. diphenhydramine, promethazine), corticosteroids (e.g. dexamethasone),
antimuscarinics, neuroleptics, AMPA antagonists, neurokinin antagonists, NO-synthase inhibitors, alpha adrenergic agonists and alpha adrenergic antagonists,
and further pharmaceutically active substances (e.g. lidocaine (lignocaine), pizotifen, isometheptene)
or the physiologically acceptable salts thereof as well as the combinations thereof.

Preferably such other substances for therapeutic, e.g. acute, treatment are acetylsalicylic acid, ibuprofen, fenoprofen, flurbiprofen, ketoprofen, dexketoprofen, naproxen, naproxen sodium, acetaminophen, diclofenac, indomethacin, ketorolac, the combination of acetylsalicylic acid with acetaminophen and caffeine, sumatriptan, zolmitriptan, naratriptan, rizatriptan, eletriptan, frovatriptan, almotriptan, the combination of sumatriptan with naproxen or naproxen sodium, ergotamine, dihydroergotamine, lasmiditan, ubrogepant, rimegepant, metoclopramide, prochlorperazine, chlorpromazine, diphenhydramine, droperidol and dexamethasone, most preferably acetylsalicylic acid, ibuprofen, naproxen, acetaminophen, diclofenac, sumatriptan, zolmitriptan, naratriptan, rizatriptan, eletriptan, frovatriptan, almotriptan, the combination of sumatriptan with naproxen or naproxen sodium, ergotamine, dihydroergotamine, lasmiditan, metoclopramide, prochlorperazine, chlorpromazine, diphenhydramine and dexamethasone.

Thus, according to one embodiment of the present invention, the SGLT2 inhibitor for use as defined hereinbefore or hereinafter is provided for use in combination with substances for preventive treatment of headache disorders, preferably selected from the group consisting of beta-adrenergic blockers, antiepileptics or anticonvulsants, antidepressants, Ca channel antagonists, CGRP antagonists, monoclonal antibodies against CGRP and its receptor, monoclonal antibodies against PACAP 38 and its receptor PAC1, angiotensin-II antagonists, histamine-H1-receptor antagonists, alpha adrenergic agonists, alpha adrenergic antagonists, NO-synthase inhibitors, and neuroleptics.

According to a preferred embodiment, the SGLT2 inhibitor for use as defined hereinbefore or hereinafter is provided for use in combination with substances for preventive treatment of headache disorders selected from the group consisting of propanolol, timolol, metoprolol, atenolol, nadolol, divalproex sodium, topiramate, gabapentin, amitriptylin, bupropion, fluoxetin, nimodipine, flunarizine, rimegepant, atogepant, eptinezumab, galcanezumab, fremanezumab, erenumab, botulinum toxin and verapamil.

According to another embodiment, the SGLT2 inhibitor for use as defined hereinbefore or hereinafter is provided for use in combination with substances for therapeutic, e.g. acute, treatment of headache disorders, preferably selected from analgesics and NSAIDs, ergot derivatives, serotonin 5-HT_{1B/1D} agonists like triptans, serotonin 5-HT_{1F} agonists, glutamate receptor antagonists, CGRP antagonists, antiemetic or prokinetic drugs, histamine-H1-receptor antagonists, corticosteroids, antimuscarinics, neuroleptics, AMPA antagonists, neurokinin antagonists, NO-synthase inhibitors, alpha adrenergic agonists and alpha adrenergic antagonists.

According to a preferred embodiment, the SGLT2 inhibitor for use as defined hereinbefore or hereinafter is provided for use in combination with substances for therapeutic, e.g. acute, treatment of headache disorders selected from the group consisting of acetylsalicylic acid, ibuprofen, fenoprofen, flurbiprofen, ketoprofen, dexketoprofen, naproxen, naproxen sodium, acetaminophen, diclofenac, indomethacin, ketorolac, the combination of acetylsalicylic acid with acetaminophen and caffeine, sumatriptan, zolmitriptan, naratriptan, rizatriptan, eletriptan, frovatriptan, almotriptan, the combination of sumatriptan with naproxen or naproxen sodium, ergotamine, dihydroergotamine, lasmiditan, ubrogepant, rimegepant, metoclopramide, prochlorperazine, chlorpromazine, diphenhydramine, droperidol and dexamethasone.

According to another embodiment, the SGLT2 inhibitor for use as defined hereinbefore or hereinafter is provided for use in combination with said substances for therapeutic, e.g. acute, treatment of headache disorders wherein the combined use is simultaneous.

According to another embodiment, the SGLT2 inhibitor for use as defined hereinbefore or hereinafter is provided for use in combination with said substances for therapeutic, e.g. acute, treatment of headache disorders wherein the combined use is sequential.

In a second aspect of the present invention, it is found that pharmaceutical compositions of SGLT2 inhibitors can be formulated that are suitable for the administration of therapeutically effective amounts of said inhibitors for the preventive and/or therapeutic, e.g. acute, treatment of headache disorders.

These pharmaceutical compositions may show advantageous effects for the treatment of headache disorders, e.g. with respect to efficacy, dosage, dose strength, dose frequency, pharmacodynamic properties, pharmacokinetic properties, fewer adverse events, convenience, compliance, etc.

Said pharmaceutical compositions may be administered by any appropriate route, e.g. via oral, buccal, sublingual, nasal, parenteral, inhalative, transdermal, subcutaneous, intravenous, intramuscular, rectal, topical, intraocular, intravitreal or intraperitoneal administration. They may be in liquid or solid form or in a form suitable for administration by inhalation or insufflation. Solid compositions for oral administration are preferred.

Suitable compositions for administering the active pharmaceutical ingredients of the present invention will be apparent to those with ordinary skill in the art and include for example tablets like oral tablets, oral dispersible tablets, effervescent tablets, pills, capsules, suppositories, lozenges, troches, nasal sprays, solutions, suspensions, syrups, elixirs, injectables, inhalatives, powders granules, etc. Suitable tablets may be obtained, for example, by mixing one or more of the above-mentioned active pharmaceutical ingredients with known excipients, for example inert diluents, carriers, disintegrants, adjuvants, surfactants, binders and/or lubricants. Film-coated tablets are most preferred. Particular pharmaceutical compositions of empagliflozin are described for example in WO 2010/092126. Likewise, suitable compositions of the other exemplary SGLT2 inhibitors mentioned above are known to the one skilled in the art

The compositions provide a therapeutically effective amount of the SGLT2 inhibitors for use according to the present invention. For instance, the composition for oral administration to a patient in need thereof may comprise a dose of the SGLT2 inhibitor in the range from 1 mg to 300 mg, in particular from 1 mg to 25 mg, e.g. 1 mg, 2.5 mg, 5 mg, 10 mg, 12.5 mg, 15 mg, 25 mg, 100 mg and 300 mg, preferably 2.5 mg, 5 mg, 10 mg and 25 mg. The composition is applied once, twice or thrice daily, preferably once daily, in particular in the case of preventive treatment.

If the method of treatment comprises co-administration of the above-mentioned SGLT2 inhibitor with other pharmaceutically active substances, it is possible to administer a pharmaceutical composition comprising not only the SGLT2 inhibitor, but also further suitable substances as mentioned above.

Alternatively, the SGLT2 inhibitor and its combination partners may be present in more than one separate pharmaceutical composition. These separate compositions may be administered together or separately, sequentially or simultaneously, jointly or in a time-shifted manner.

Further, said one or more pharmaceutical compositions may be contained within a pharmaceutical kit for simultaneous, or sequential use of effective doses of the above-mentioned active pharmaceutical ingredients. For instance, said pharmaceutical kit may encompass one or more of the above-mentioned compositions, and optionally devices for their application, e.g. in separate compartments.

Thus, according to one embodiment of the second aspect of the present invention, a pharmaceutical composition is provided that comprises therapeutically effective amounts of one or more of the above-mentioned SGLT2 inhibitors, and one or more pharmaceutically acceptable excipients for use in a method for the preventive and/or therapeutic, e.g. acute, treatment of headache disorders.

Preferably, the composition comprises therapeutically effective amounts of empagliflozin; preferably, the treatment is preventive treatment; preferably, the headache disorder is migraine, selected from migraine without aura, migraine with aura, chronic migraine, complications of migraine (e.g. status migrainosus), probable migraine, and episodic syndromes that may be associated with migraine.

According to another embodiment, the pharmaceutical composition as defined hereinbefore or hereinafter is selected from compositions for oral administration, preferably from capsules and tablets, most preferably from film-coated tablets.

According to another embodiment, the pharmaceutical composition, preferably a film-coated tablet, as defined hereinbefore or hereinafter comprises the SGLT2 inhibitor in an amount in the range from 1 mg to 300 mg, in particular from 1 mg to 25 mg, preferably 2.5 mg, 5 mg, 10 mg or 25 mg.

According to another embodiment, the pharmaceutical composition as defined hereinbefore or hereinafter additionally comprises therapeutically effective amounts of one or more, preferably one, further pharmaceutically active substances, as mentioned above, suitable for the treatment of headache disorders.

According to another embodiment, a pharmaceutical kit for the treatment of headache disorders is provided that comprises one or more pharmaceutical compositions as defined hereinbefore or hereinafter, for simultaneous, sequential and/or separate use of the active ingredients, and optionally a medical device for their administration.

Preferably, said kit comprises a first compartment containing a pharmaceutical composition of one or more SGLT2 inhibitors as defined hereinbefore or hereinafter, a second compartment containing a pharmaceutical composition of another pharmaceutically active substance suitable for the treatment of headache disorders as defined hereinbefore or hereinafter, and optionally a third compartment containing a medical device for administration of the contents of the first and/or second compartment; preferably said kit is for simultaneous, sequential and/or separate use of said active ingredients.

In a third aspect of the present invention, a method for the treatment of headache disorders in a patient in need thereof with one or more of the above-mentioned SGLT2 inhibitors is described. Furthermore, the present invention relates to a method for the treatment of headache disorders with one or more of the above-mentioned pharmaceutical compositions.

Said method is characterized by the features and embodiments described above for the first and second aspects of the present invention.

In a fourth aspect of the present invention, the use of the above-mentioned SGLT2 inhibitors in the manufacture of a medicament for the above-mentioned method for the treatment of headache disorders in a patient in need thereof is described.

Said medicament and said method are characterized by the features and embodiments described above for the first, second and third aspects of the present invention.

Further embodiments and features of the present invention may become apparent from the following examples.

### EXAMPLES AND EXPERIMENTAL DATA

The following examples are for the purpose of illustration of the principles of the invention only and are not intended in any way to limit the scope of the present invention.

### Example 1: Treatment of Patients with Migraine

The efficacy of treatment with empagliflozin in a relevant population of patients with migraine is investigated in a randomised, double-blind, placebo controlled, parallel group trial to compare treatment with empagliflozin with placebo as add-on therapy to standard of care. The duration of the patients follow-up is preferably a long-term treatment, for example 24, 48, or 52 weeks.

Patients include adult individuals with migraine. Main inclusion criteria are high risk for migraine, possibly enriched with metabolic risk factors.

Empagliflozin is administered to patients once daily, e.g. as a 2.5 mg and/or a 10 mg and/or a 25 mg oral dose. The primary endpoint of the study is duration and/or count of migraine episodes.

The key secondary endpoint relates to the use of analgesic medication, sick days, medical consultations, and/or hospitalization.

Further secondary endpoints are quality of life endpoints.

Further endpoints may relate to measurement of ketone levels.

Safety criteria comprise blood pressure, heart rate and adverse events.

### Example 2: Empagliflozin Film-Coated Tablets

| | 2.5 mg/ | 5 mg/ | 10 mg/ | 25 mg/ |
|---|---|---|---|---|
| Active substance | per tablet | per tablet | per tablet | per tablet |
| Wet granulation | | | | |
| Empagliflozin | 2.5000 | 5.000 | 10.00 | 25.00 |
| Lactose Monohydrate | 40.6250 | 81.250 | 162.50 | 113.00 |
| Microcrystalline | 12.5000 | 25.000 | 50.00 | 40.00 |
| Cellulose | | | | |
| Hydroxypropyl | 1.8750 | 3.750 | 7.50 | 6.00 |
| Cellulose | | | | |
| Croscarmellose Sodium | 1.2500 | 2.500 | 5.00 | 4.00 |
| Purified Water | q.s. | q.s. | q.s. | q.s. |
| Dry Adds | | | | |
| Microcrystalline | 3.1250 | 6.250 | 12.50 | 10.00 |
| Cellulose | | | | |
| Colloidal silicon | 0.3125 | 0.625 | 1.25 | 1.00 |
| dioxide | | | | |
| Magnesium stearate | 0.3125 | 0.625 | 1.25 | 1.00 |
| Total core | 62.5000 | 125.000 | 250.00 | 200.00 |
| Film Coating | | | | |
| Film coating system | 2.5000 | 4.000 | 7.00 | 6.00 |
| Purified Water | q.s. | q.s. | q.s. | q.s. |
| Total | 65.000 | 129.000 | 257.00 | 206.00 |

Amounts are given in mg per film-coated tablets. The term “active substance” denotes empagliflozin, especially its crystalline form as described in WO 2006/117359 and WO 2011/039107.

Details regarding the manufacture of the tablets, the active pharmaceutical ingredient, the excipients and the film-coating system are described in WO 2010/092126, in particular in the Examples 5 and 6, which hereby is incorporated herein in its entirety. WO 2010/092126 also discloses further examples of compositions and dosage forms for oral administration.

1. A method for treating headache disorders, the method comprising administering a therapeutically effective amount of an SGLT2 inhibitor to a patient in need thereof.

2. The method according to claim 1 wherein the inhibitor is empagliflozin.

3. The method according to claim 1 wherein the treatment is preventive treatment.

4. The method according to claim 1 wherein the headache disorder is a primary headache disorder selected from migraine, tension-type headache, and trigeminal autonomic cephalalgias.

5. The method according to claim 1 wherein the headache disorder is migraine selected from migraine without aura, migraine with aura, chronic migraine, complications of migraine, probable migraine, and episodic syndromes that may be associated with migraine.

6. The method according to claim 2 wherein the inhibitor is administered orally in a once-daily dose of 2.5 mg, 5 mg, 10 mg or 25 mg.

7. The method according to claim 1 wherein the inhibitor is administered simultaneously or sequentially in combination with one or more pharmaceutically active substances for treatment of headache disorders, selected from the group consisting of beta-adrenergic blockers, antiepileptics or anticonvulsants, antidepressants, Ca channel antagonists, CGRP antagonists, monoclonal antibodies against CGRP and its receptor, monoclonal antibodies against PACAP 38 and its receptor PAC1, angiotensin-II antagonists, histamine-H1-receptor antagonists, alpha adrenergic agonists, alpha adrenergic antagonists, NO-synthase inhibitors, neuroleptics, analgesics and NSAIDs, ergot derivatives, serotonin 5-HT_{1B/1D} agonists, serotonin 5-HT_{1F} agonists, glutamate receptor antagonists, antiemetic or prokinetic drugs, corticosteroids, antimuscarinics, AMPA antagonists, and neurokinin antagonists.

8. The method according to claim 1 wherein the inhibitor is administered simultaneously or sequentially in combination with one or more pharmaceutically active substances selected from the group consisting of propanolol, timolol, metoprolol, atenolol, nadolol, divalproex sodium, topiramate, gabapentin, amitriptylin, bupropion, fluoxetin, nimodipine, flunarizine, rimegepant, atogepant, eptinezumab, galcanezumab, fremanezumab, erenumab, botulinum toxin and verapamil.

9. A method for treating headache disorders, the method comprising administering to a patient in need thereof a pharmaceutical composition comprising a therapeutically effective amount of one or more SGLT2 inhibitors and one or more pharmaceutically acceptable excipients.

10. The method according to claim 9 wherein the SGLT2 inhibitor is empagliflozin and wherein the treatment of headache disorders is the preventive treatment of migraine, selected from migraine without aura, migraine with aura, chronic migraine, complications of migraine, probable migraine, and episodic syndromes that may be associated with migraine.

11. The method according to claim 9 wherein the composition is an oral composition.

12. The method pharmaceutical according to claim 9 wherein the composition comprises empagliflozin in an amount of 2.5 mg, 5 mg, 10 mg or 25 mg.

13. The method according to claim 9 wherein the composition additionally comprises one or more further pharmaceutically active substances selected from the group consisting of propanolol, timolol, metoprolol, atenolol, nadolol, divalproex sodium, topiramate, gabapentin, amitriptylin, bupropion, fluoxetin, nimodipine, flunarizine, rimegepant, atogepant, eptinezumab, galcanezumab, fremanezumab, erenumab, botulinum toxin and verapamil.

14. (canceled)

15. (canceled)

16. The method according to claim 11, wherein the oral composition is in the form of a tablet.
